(19) United States
(12) Patent Application Publication
(10) Pub. No.: US 2006/0090235 A1
(43) Pub. Date: May 4, 2006

(54) Modular headwear assembly

(76) Inventor: Andrew J. Clark, San Antonio, TX (US)

Correspondence Address: ASHKAN NAJAFI, P.A., 6817 SOUTHPOINT PARKWAY, SUITE 2301, JACKSONVILLE, FL, 32216, US

(21) Appl. No.: 10/974,901

(22) Filed: Oct. 28, 2004

(51) Int. Cl. A61F 9/00

(52) U.S. Cl. 2/12

(57) ABSTRACT

A disposable headwear assembly for protecting a user from harmful sunlight includes a box-like structure with perforated segments that a user can detach from the structure. The segments include at least one peak portion and one band portion. Such peak and band portions are shaped for interlocking together to form a visor. In a preferred embodiment the band is provided with locking slots for receiving the flange portions of the peak. Such a band further has a locking tab for mating with the other slots. An alternate embodiment of the band includes two interlocking segments. The first segment has slots for receiving the peak. The second segment has locking slots for cooperating with the locking tabs on the first segment.

### CROSS REFERENCE TO RELATED APPLICATIONS

Not Applicable.

### STATEMENT REGARDING FEDERALLY SPONSORED RESEARCH OR DEVELOPMENT

Not Applicable.

### REFERENCE TO A MICROFICHE APPENDIX

Not Applicable.

### BACKGROUND OF THE INVENTION

1. Technical Field

This invention relates to a protective visor and, more particularly, to a decorative protective visor that may be discarded after use.

2. Prior Art

Outdoor activities create differing problems in regard to the comfort and suitability of sunglasses as may be needed by a participant to protect the eyes from the effects of sunlight. In a typical physically active activity, occurring in sunlight which may negatively affect vision, the participant may be perspiring about the face and head, and the wearing of commonly available nose and ear supported sunglasses becomes problematic. Sunglasses slip from their common mounting places on sweaty noses and ears, requiring constant re-adjustment, complicated by perspiration forming along the participant's brow and/or hands which may fog or otherwise be transferred onto the sunglasses lens, obscuring vision. Running, jumping and other jarring competitive activities only increase the problems and, particularly in situations wherein the participant is required to use his hands during the sporting activity, may disrupt the participant's ability to compete.

Devices adapted to be worn on the forehead such as caps and visors have existed for hundreds of years and for reasons ranging from basic eye shading to status symbols, fashion statements and advertising media. Unfortunately, a number of drawbacks, difficulties and annoying inconveniences have also existed, including the following: trying to find a cap or visor that fits correctly; trying to find one that is comfortable; trying to find one that is durable in use, that can be occasionally cleaned without deformation or damage; and trying to find one that has all of the desired characteristics such as color, design, and the correct athletic team symbols or other insignia.

The prior art shows many head coverings. Most are designed for a single kind of use. Consequently, a person who wears hats needs many of them in order to be prepared for all eventualities. This is particularly true for people who work or recreate in the out-of-doors. The alternative to owning several caps or visors is owning just a few and washing them in the washing machine. This often leads to the breakdown of the plastic and material parts of the head covering.

Accordingly, a need remains for a visor apparatus that provides adequate protection from sunlight and is convenient to use. The present invention satisfies such a need by providing a disposable visor that is durable and attractive. A user can enjoy the outdoors knowing their face is being protected from the sun, go home and simply dispose of the visor. The structural integrity of the visor is never in jeopardy from going through the washing machine.

### BRIEF SUMMARY OF THE INVENTION

In view of the foregoing background, it is therefore an object of the present invention to provide a disposable headwear assembly for protecting a user from harmful sunlight. These and other objects, features, and advantages of the invention include a selected quantity of stock material that has a box-like structure and is formed from sunlight-impermeable material. Such a structure includes a plurality of sides respectively including a plurality of perforated segments such that a user can readily detach the segments from the structure.

The segments each include at least one of a peak portion and a band portion respectively. Such peak and band portions are resiliently shaped for interlocking together and forming a general visor shape such that the peak and band sections can be adjustably positioned about a user's head while effectively maintaining a locked position. The sunlight-impermeable material is formed from cardboard, or some other comparable material well-known to those skilled in the industry. Each of the segments is provided with decorative surface indicia for illustrating a predetermined theme.

The peak portion is preferably sized and shaped to form a visor-like member including a plurality of integrally disposed flange portions that have a generally T-shape. Such a peak portion further has a substantially arcuate proximal edge and is abutted against the user's head.

The band portion is preferably sized and shaped to form an elongated and flexible strap removably engageable with the peak portion such that the peak portion becomes situated distally of the band portion. The band portion has opposed end portions adaptable between linear and arcuate positions such that the end portions thereof can be locked at a fixed position.

Furthermore, in a preferred embodiment the band portion is provided with a plurality of locking slots disposed along a length thereof. Selected ones of the slots are for receiving the flange portions therethrough. Such a band portion further has a locking tab for selectively mating with other ones of the slots.

Advantageously, in an alternate embodiment the band portion includes a first segment and a second segment adjustably lockable therewith. Such first and second segments are positionable against front and rear hemispheres of the user's head such that a mating location becomes positioned adjacent a user's ears respectively. The first segment is provided with a plurality of slots selectively spaced therealong and further has oppositely disposed locking tabs integrally formed therewith. The first segment has a centrally disposed portion that has a raised upper edge aligned adjacent to the peak portion. The second segment has a plurality of locking slots formed therein for cooperating with the locking tabs such that the band portion can be adapted to a generally annular shape. The peak portion includes locking tabs respectively engageable with corresponding ones of the first segment locking slots such that the peak portion extends distally away from the band portion to protect the user from sunlight.

### BRIEF DESCRIPTION OF THE SEVERAL VIEWS OF THE DRAWING

The novel features believed to be characteristic of this invention are set forth with particularity in the appended claims. The invention itself, however, both as to its organization and method of operation, together with further objects and advantages thereof, may best be understood by reference to the following description taken in connection with the accompanying drawings in which:

FIG. 1 is a perspective view showing a disposable headwear assembly, in accordance with the present invention;

FIG. 1A is a front elevational view showing the band portion of the present invention shown in FIG. 1;

FIG. 2 is an exploded perspective view showing the band and visor of the present invention shown in FIG. 1;

FIG. 3 is a perspective view of the visor shown in FIG. 1;

FIG. 4 is a perspective view showing an alternate embodiment of the box of the present invention;

FIG. 5 is an exploded view showing an alternate embodiment of the band and visor shown in FIG. 1; and

FIG. 6 is a perspective view showing the alternate embodiment of the visor assembly shown in FIG. 5.

### DETAILED DESCRIPTION OF THE INVENTION

The present invention will now be described more fully hereinafter with reference to the accompanying drawings, in which preferred embodiments of the invention is shown. This invention may, however, be embodied in many different forms and should not be construed as limited to the embodiments set forth herein. Rather, these embodiments are provided so that this application will be thorough and complete, and will fully convey the true scope of the invention to those skilled in the art. Like numbers and primes refer to like elements throughout the figures.

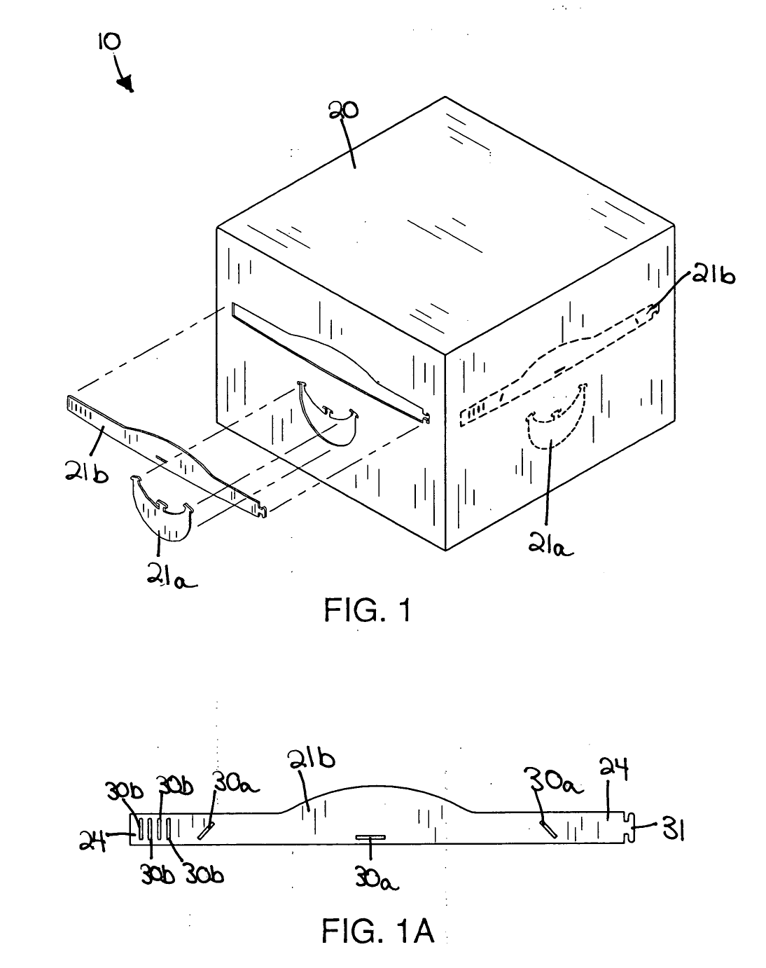

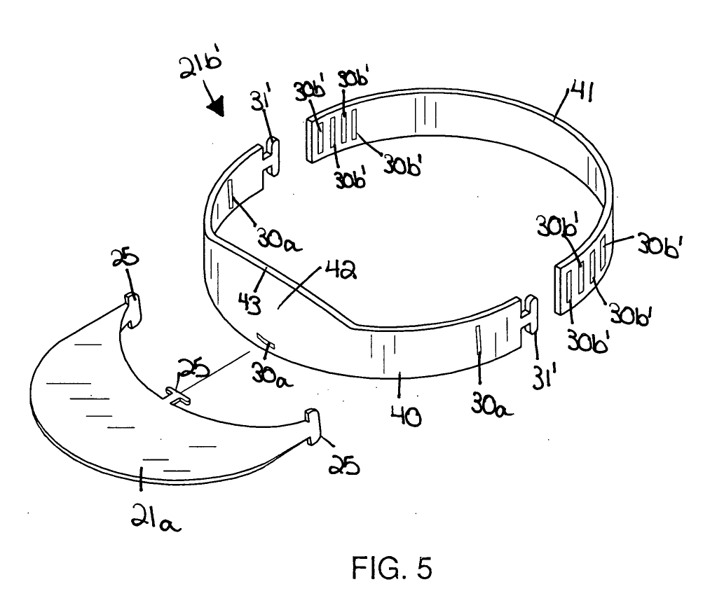

The assembly of this invention is referred to generally in FIGS. 1-6 by the reference numeral 10 and is intended to protect a user's faces from the sun. It should be understood that the assembly 10 may be produced in a variety of colors and designs and may be used by people of all ages.

Initially referring to FIG. 1, the assembly 10 includes a selected quantity of stock material that has a box-like structure 20 and is formed from sunlight-impermeable material. Such a structure 20 includes a plurality of sides respectively including a plurality of perforated segments 21 such that a user can readily detach the segments 21 from the structure 20. The segments 21 each include at least one of a peak portion 21a and a band portion 21b respectively. Such peak 21a and band 21b portions are resiliently shaped for interlocking together and forming a general visor shape such that the peak 21a and band 21b sections can be adjustably positioned about a user's head while effectively maintaining a locked position. The sunlight-impermeable material is preferably formed from cardboard. Of course, the assembly 10 may be formed from other well known materials having properties suitable for the intended application. Each of the segments 21 is provided with decorative surface indicia (not shown) for illustrating a predetermined theme.

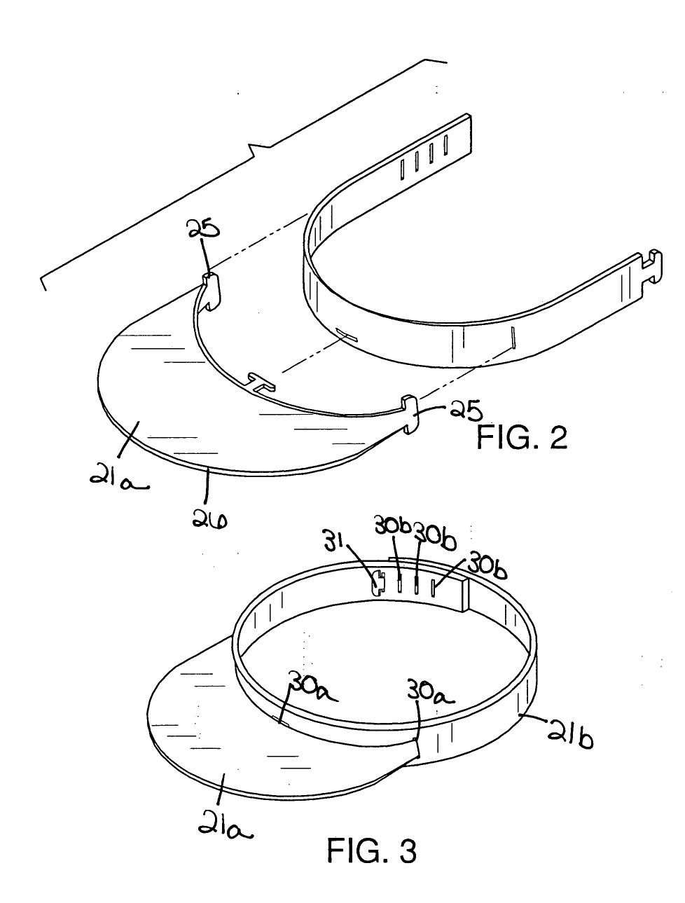

Now referring to FIG. 2, the peak portion 21a is preferably sized and shaped to form a visor-like member including a plurality of integrally disposed flange portions 25 that have a generally T-shape. Such a peak portion 21a further has a substantially arcuate proximal edge 26 and is abutted against the user's head.

Referring to FIG. 1a, the band portion 21b is preferably sized and shaped to form an elongated and flexible strap removably engageable with the peak portion 21a such that the peak portion 21a becomes situated distally of the band portion 21b. The band portion 21b has opposed end portions 24 adaptable between linear and arcuate positions such that the end portions 24 thereof can be locked at a fixed position.

Continuing to refer to FIG. 1a as well as FIG. 3, in a preferred embodiment the band portion 21b is provided with a plurality of locking slots 30 disposed along a length thereof. Selected ones 30a of the slots 30 are for receiving the flange portions 25 therethrough. Such a band portion 21b further has a locking tab 31 for selectively mating with other ones 30b of the slots 30.

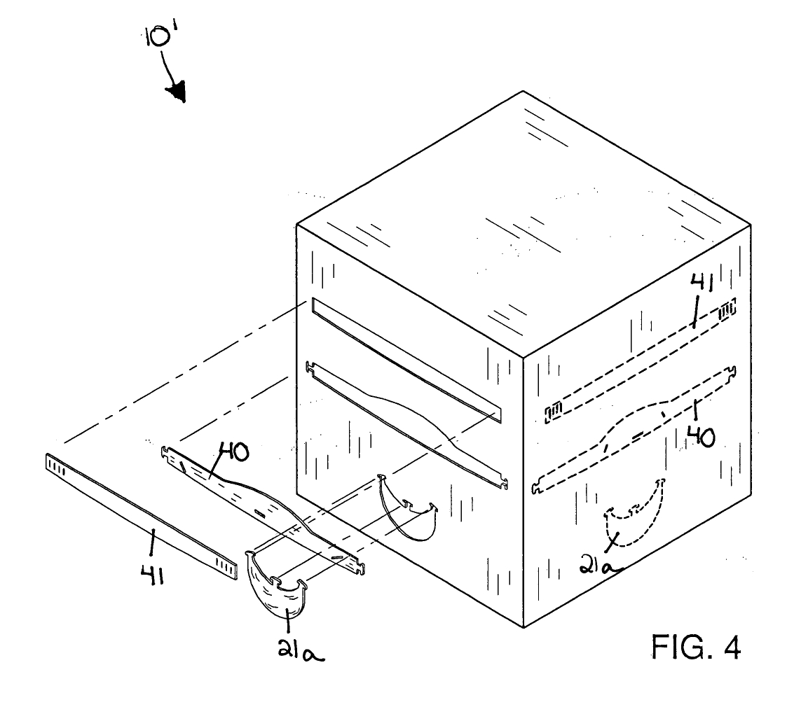

Now referring to an alternate embodiment 10′ in FIGS. 4-6, the band portion 21b′ advantageously includes a first segment 40 and a second segment 41 adjustably lockable therewith. Such first 40 and second 41 segments are positionable against front and rear hemispheres of the user's head such that a mating location becomes positioned adjacent a user's ears respectively. The first segment 40 is provided with a plurality of slots 30a selectively spaced therealong and further has oppositely disposed locking tabs 31′ integrally formed therewith.

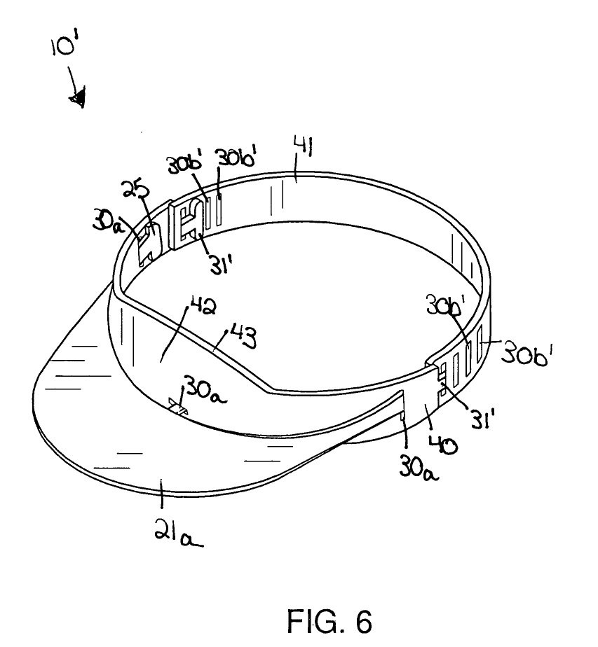

The first segment 40 may also have a centrally disposed portion 42 that has a raised upper edge 43 aligned adjacent to the peak portion 21a. The second segment 41 has a plurality of locking slots 30b′ formed therein for cooperating with the locking tabs 31′ such that the band portion 21b′ can be adapted to a generally annular shape. The peak portion 21a includes locking tabs 25 respectively engageable with corresponding ones of the first segment locking slots 30a such that the peak portion 21a extends distally away from the band portion 21b′ to protect the user from sunlight, best shown in FIG. 6.

While the invention has been described with respect to a certain specific embodiments, it will be appreciated that many modifications and changes may be made by those skilled in the art without departing from the spirit of the invention. It is intended, therefore, by the appended claims to cover all such modifications and changes as fall within the true spirit and scope of the invention.

In particular, with respect to the above description, it is to be realized that the optimum dimensional relationships for the parts of the present invention may include variations in size, materials, shape, form, function and manner of operation. The assembly and use of the present invention are deemed readily apparent and obvious to one skilled in the art.

What is claimed as new and what is desired to secure by Letters Patent of the United States is:

1. A disposable headwear assembly for protecting a user from harmful sunlight, said assembly comprising:
a selected quantity of stock material having a box-like structure and being formed from sunlight-impermeable material, said structure including a plurality of sides respectively comprising a plurality of perforated segments such that a user can readily detach said segments from said structure, said segments each comprising at least one of a peak portion and a band portion respectively, said peak and band portions being resiliently shaped for interlocking together and forming a general visor shape such that said peak and band sections can be adjustably positioned about a user's head while being effectively maintained a locked position.

2. The headwear assembly of claim 1, wherein said peak portion is sized and shaped to form a visor-like member including a plurality of integrally disposed flange portions having generally T-shapes, said peak portion further having a substantially arcuate proximal edge for being abutted against the user's head.

3. The headwear assembly of claim 2, wherein said band portion is sized and shaped to form an elongated and flexible strap removably engageable with said peak portion such that said peak portion becomes situated distally of said band portion, said band portion having opposed end portions adaptable between linear and arcuate positions such that said end portions thereof can be locked at a fixed position.

4. The headwear assembly of claim 3, wherein said band portion is provided with a plurality of locking slots disposed along a length thereof, selected ones of said slots for receiving said flange portions therethrough, said band portion further having a locking tab for selectively mating with other ones of said slots.

5. The headwear assembly of claim 1, wherein said band portion comprises: a first segment and a second segment adjustably lockable therewith, said first and second segments being positionable against front and rear hemispheres of the user's head such that a mating location becomes positioned adjacent a user's ears respectively, said first segment being provided with a plurality of slots selectively spaced therealong and further having oppositely disposed locking tabs integrally formed therewith, said second segment having a plurality of locking slots formed therein for cooperating with said locking tabs such that said band portion can be adapted to a generally annular shape, said peak portion including locking tabs respectively engageable with corresponding ones of said first segment locking slots such that said peak portion extends distally away from said band portion to protect the user from sunlight.

6. The headwear assembly of claim 5, wherein said first segment has a centrally disposed portion having a raised upper edge aligned adjacent to said peak portion.

7. A disposable headwear assembly for protecting a user from harmful sunlight, said assembly comprising:
a selected quantity of stock material having a box-like structure and being formed from sunlight-impermeable material, said structure including a plurality of sides respectively comprising a plurality of perforated segments such that a user can readily detach said segments from said structure, said segments each comprising at least one of a peak portion and a band portion respectively, said peak and band portions being resiliently shaped for interlocking together and forming a general visor shape such that said peak and band sections can be adjustably positioned about a user's head while being effectively maintained a locked position, said sunlight-impermeable material being formed from cardboard.

8. The headwear assembly of claim 7, wherein said peak portion is sized and shaped to form a visor-like member including a plurality of integrally disposed flange portions having generally T-shapes, said peak portion further having a substantially arcuate proximal edge for being abutted against the user's head.

9. The headwear assembly of claim 8, wherein said band portion is sized and shaped to form an elongated and flexible strap removably engageable with said peak portion such that said peak portion becomes situated distally of said band portion, said band portion having opposed end portions adaptable between linear and arcuate positions such that said end portions thereof can be locked at a fixed position.

10. The headwear assembly of claim 9, wherein said band portion is provided with a plurality of locking slots disposed along a length thereof, selected ones of said slots for receiving said flange portions therethrough, said band portion further having a locking tab for selectively mating with other ones of said slots.

11. The headwear assembly of claim 7, wherein said band portion comprises: a first segment and a second segment adjustably lockable therewith, said first and second segments being positionable against front and rear hemispheres of the user's head such that a mating location becomes positioned adjacent a user's ears respectively, said first segment being provided with a plurality of slots selectively spaced therealong and further having oppositely disposed locking tabs integrally formed therewith, said second segment having a plurality of locking slots formed therein for cooperating with said locking tabs such that said band portion can be adapted to a generally annular shape, said peak portion including locking tabs respectively engageable with corresponding ones of said first segment locking slots such that said peak portion extends distally away from said band portion to protect the user from sunlight.

12. The headwear assembly of claim 11, wherein said first segment has a centrally disposed portion having a raised upper edge aligned adjacent to said peak portion.

13. A disposable headwear assembly for protecting a user from harmful sunlight, said assembly comprising:
a selected quantity of stock material having a box-like structure and being formed from sunlight-impermeable material, said structure including a plurality of sides respectively comprising a plurality of perforated segments such that a user can readily detach said segments from said structure, said segments each comprising at least one of a peak portion and a band portion respectively, said peak and band portions being resiliently shaped for interlocking together and forming a general visor shape such that said peak and band sections can be adjustably positioned about a user's head while being effectively maintained a locked position, said sunlight-impermeable material being formed from cardboard, each said segments being provided with decorative surface indicia for illustrating a predetermined theme.

14. The headwear assembly of claim 13, wherein said peak portion is sized and shaped to form a visor-like member including a plurality of integrally disposed flange portions having generally T-shapes, said peak portion further having a substantially arcuate proximal edge for being abutted against the user's head.

15. The headwear assembly of claim 14, wherein said band portion is sized and shaped to form an elongated and flexible strap removably engageable with said peak portion such that said peak portion becomes situated distally of said band portion, said band portion having opposed end portions adaptable between linear and arcuate positions such that said end portions thereof can be locked at a fixed position.

16. The headwear assembly of claim 15, wherein said band portion is provided with a plurality of locking slots disposed along a length thereof, selected ones of said slots for receiving said flange portions therethrough, said band portion further having a locking tab for selectively mating with other ones of said slots.

17. The headwear assembly of claim 13, wherein said band portion comprises: a first segment and a second segment adjustably lockable therewith, said first and second segments being positionable against front and rear hemispheres of the user's head such that a mating location becomes positioned adjacent a user's ears respectively, said first segment being provided with a plurality of slots selectively spaced therealong and further having oppositely disposed locking tabs integrally formed therewith, said second segment having a plurality of locking slots formed therein for cooperating with said locking tabs such that said band portion can be adapted to a generally annular shape, said peak portion including locking tabs respectively engageable with corresponding ones of said first segment locking slots such that said peak portion extends distally away from said band portion to protect the user from sunlight.

18. The headwear assembly of claim 17, wherein said first segment has a centrally disposed portion having a raised upper edge aligned adjacent to said peak portion.
